(19) United States
(12) Patent Application Publication
(10) Pub. No.: US 2013/0042871 A1
(43) Pub. Date: Feb. 21, 2013

(54) Breathing Assistance Apparatus

(71) Applicant: ERIC CHANG, TAICHUNG CITY (TW)

(72) Inventor: ERIC CHANG, TAICHUNG CITY (TW)

(73) Assignee: HSINER CO, LTD., Taichung City (TW)

(21) Appl. No.: 13/354,397

(22) Filed: Jan. 20, 2012

(30) Foreign Application Priority Data
Aug. 15, 2011 (TW) 100129085

(51) Int. Cl. A61M 16/06; A61M 15/08

(52) U.S. Cl. 128/205.25

(57) ABSTRACT

A breathing assistance apparatus is used to deliver gases to a wearer. The breathing assistance apparatus includes: a main body unit having a main body and two side straps connected to the main body, each of the side straps having a strap body, at least one side cushion projecting from the strap body and adapted for contact with the wearer's head, and a buffering space disposed within the side cushion; a head strap unit connected to the side straps; a nasal prong unit mounted to and disposed behind the main body; and an intake unit mounted to and disposed in front of the main body.

### BACKGROUND OF THE INVENTION

1. Field of the Invention

This invention relates to medical supplies, and more particularly to a breathing assistance apparatus that includes two nasal prongs and that is adapted to deliver gases to a patient.

2. Description of the Related Art

Referring to FIG. 1, a conventional breathing assistance apparatus includes a main body unit 1 having a main body 101 and two side straps 102, a head strap unit 2 connected to the main body unit 1, a nasal prong unit 3 disposed behind and connected to the main body 101, and an intake tube 4 disposed in front of and connected to the main body 101. Although the main body unit 1 is made of silicone gel, and has a soft characteristic, when it is worn on the wearer's head during sleeping, the side straps 102 contact directly the face of the wearer, thereby resulting in the wearer's discomfort, which will affect adversely the sleeping quality.

### SUMMARY OF THE INVENTION

The object of this invention is to provide a breathing assistance apparatus that can be worn comfortably on a wearer.

According to this invention, a breathing assistance apparatus is used to deliver gases to a wearer. The breathing assistance apparatus includes: a main body unit having a main body and two side straps connected to the main body, each of the side straps having a strap body, at least one side cushion projecting from the strap body and adapted for contact with the wearer's head, and a buffering space disposed within the side cushion; a head strap unit connected to the side straps; a nasal prong unit mounted to and disposed behind the main body; and an intake unit mounted to and disposed in front of the main body.

Due to inclusion of the side cushions of the side straps in the main body unit, the breathing assistance apparatus can be worn comfortably on the wearer's head so as to improve the sleeping quality of the wearer.

### BRIEF DESCRIPTION OF THE DRAWINGS

These and other features and advantages of this invention will become apparent in the following detailed description of two preferred embodiments of this invention, with reference to the accompanying drawings, in which:

FIG. 1 is a perspective view of a conventional breathing assistance apparatus;

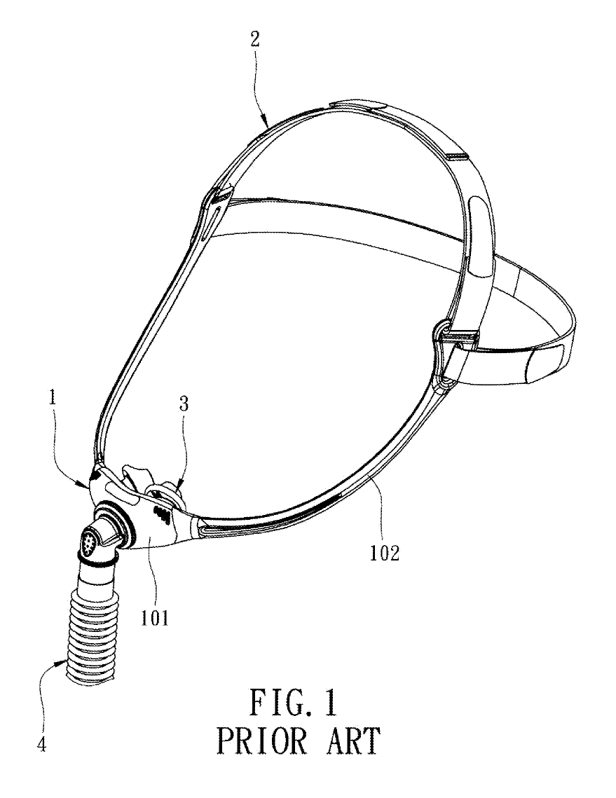

FIG. 2 is a perspective view of the first preferred embodiment of a breathing assistance apparatus according to this invention;

FIG. 3 is a fragmentary, partly sectional view of the first preferred embodiment;

FIG. 4 is a sectional view of a side cushion of a side strap of the first preferred embodiment;

FIG. 5 is a perspective view of the second preferred embodiment of a breathing assistance apparatus according to this invention;

FIG. 6 is a top view of the second preferred embodiment;

FIG. 7 is a sectional view of a side cushion of a side strap of the second preferred embodiment;

FIG. 8 is a perspective view of the third preferred embodiment of a breathing assistance apparatus according to this invention;

FIG. 9 is a fragmentary side view of the third preferred embodiment; and

FIG. 10 is a fragmentary sectional view of third preferred embodiment, illustrating a side cushion.

### DETAILED DESCRIPTION OF THE PREFERRED EMBODIMENTS

Before the present invention is described in greater detail in connection with the preferred embodiments, it should be noted that similar elements and structures are designated by like reference numerals throughout the entire disclosure.

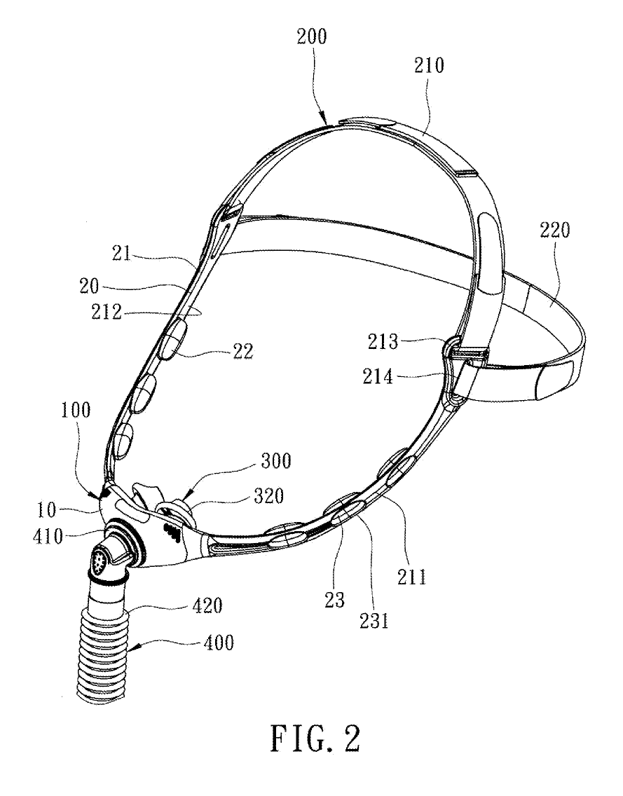

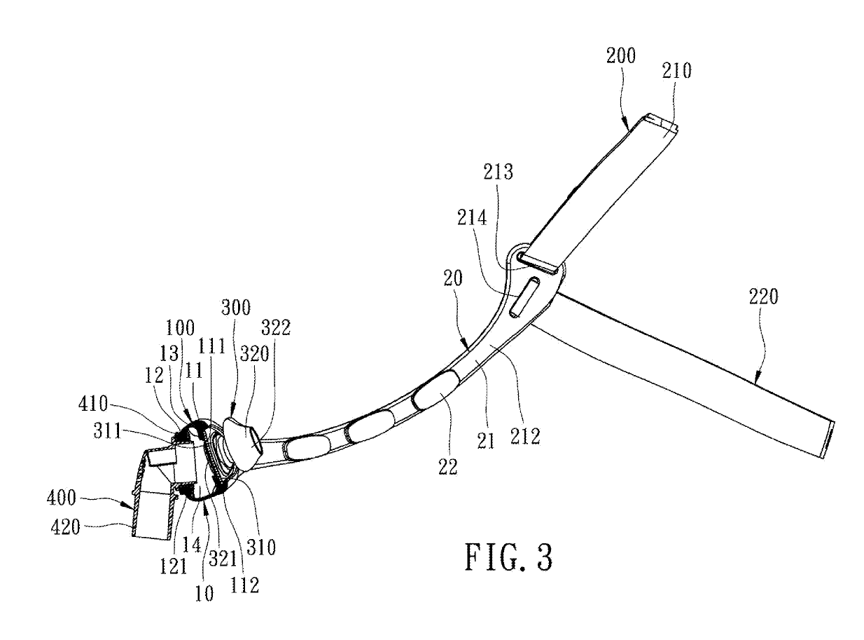

Referring to FIGS. 2 and 3, the first preferred embodiment of a breathing assistance apparatus according to this invention is used to deliver gases to the wearer (i.e., a patient). The breathing assistance apparatus includes a main body unit 100, a head strap unit 200 connected to the main body unit 100, a nasal prong unit 300 mounted to the main body unit 100, and an intake unit 400 mounted to the main body unit 500.

The main body unit 100 is made of silicone gel, and includes a main body 10 and two side straps 20 connected integrally to the main body 10. The main body 20 has a rear wall 11, a front wall 12, and a connecting wall 13 connected between the front and rear walls 12, 11 to define a chamber 14 thereamong. The rear wall 11 has an insert hole 111 in spatial communication with the chamber 19, and an insert groove 112 disposed behind and in spatial communication with the insert hole 111 and having a width that reduces outwardly. The front wall 12 has a coupling hole 121 in spatial communication with the chamber 14. In this embodiment, the insert groove 112 is formed in a rear side surface of the rear wall 11.

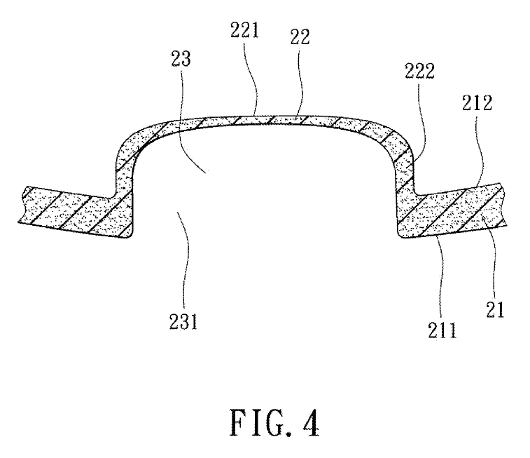

With further reference to FIG. 4, each of the side straps 20 has a strap body 21, a plurality of side cushions 22 projecting from the strap body 21 and adapted for contact with the wearer's head (not shown), a plurality of buffering spaces 23 disposed respectively within the side cushions 22, and a plurality of openings 231 formed in the strap body 21. The strap body 21 has an outer side surface 211, an inner side surface 212 opposite to the outer side surface 211, a first through hole 213 formed through an end portion of the strap body 21, and a second through hole 214 formed in the end portion of the strap body 21 and adjacent to the first through hole 213. Each of the side cushions 22 projects from the inner side surface 212, and has a head-engaging portion 221, and a loop-shaped connecting portion 222 connected integrally between an outer periphery of the head-engaging portion 221 and a wall of the corresponding side strap 21 defining the corresponding opening 231. As such, the buffering spaces 23 in the side cushions 22 are disposed respectively inside the loop-shaped connecting portions 222 of the side cushions 22, and are in spatial communication with the openings 231 in the side straps 20, respectively.

The head strap unit 200 is connected to the side straps 20 of the main body unit 100, and has a first head strap 210 extending through the first through holes 214 in the side straps 20, and a second head strap 220 extending through the second through holes 214 in the side straps 20.

The nasal prong unit 300 is disposed behind the main body 10 of the main body unit 100, and includes a main plate 310 inserted into the insert groove 112 and aligned with the insert hole 111, and two nasal prongs 320 disposed on the main plate 310 in a known manner and in spatial communication with the chamber 14. The main plate 310 is formed with two mounting holes 311. The nasal prongs 320 are made of silicone gel, and are hollow. Each of the nasal prongs 320 has an insert barrel 321 inserted into the corresponding mounting hole 311, and a nostril-engaging plug 322 inserted into the corresponding nostril of the wearer.

The intake unit 400 is disposed in front of the main body 20 of the main body unit 100, and has a hollow tube-sleeving member 410 inserted into the coupling hole 121, and an intake tube 420 having an end permitting the tube-sleeving member 410 to be sleeved fixedly thereon.

During use, the nostril-engaging plugs 322 of the nasal prong unit 300 are inserted respectively into the nostrils of the wearer, and an assembly of the first head strap 210 and the main body unit 100 is sleeved on the wearer's head, in such a manner that the first head strap 210 is adjusted in length to press against a top end of the wearer's head in a known manner, and the side cushions 22 are pressed respectively against left and right sides of the wearer's head. Subsequently, the second head strap 220 is adjusted in length to press against a rear side of the wearer's head in a known manner. As such, the side cushions 221 of the side cushions 22 abut resiliently against the wearer's head, thereby providing a comfortable feeling to the wearer, which can improve the sleeping quality of the wearer.

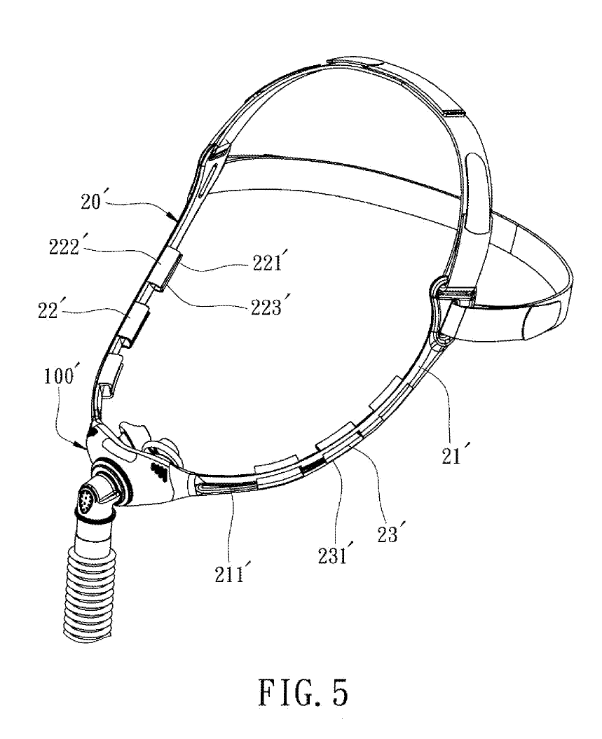

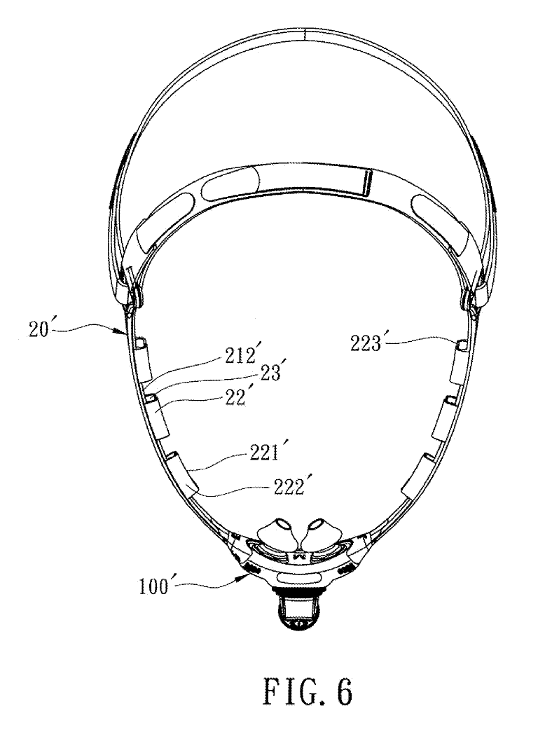

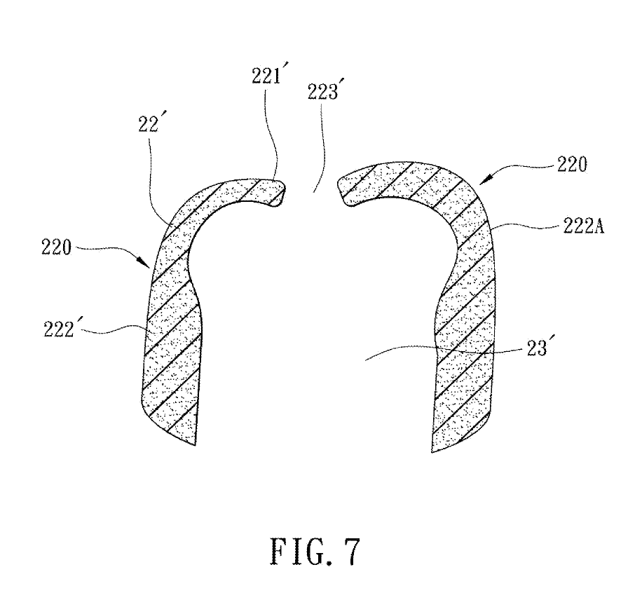

FIGS. 5, 6, and 7 show the second preferred embodiment of a breathing assistance apparatus according to this invention, which is similar in construction to the first preferred embodiment. Unlike the first preferred embodiment, the side cushion 22′ of each of side straps 20′ of the main body unit 100′ has two spaced-apart cushioning members 220. Each of the cushioning members 220 has a head-engaging plate portion 221′ and a connecting plate portion 222′. Each of the connecting plate portions 222′ of the cushioning members 220 extends integrally from a wall of the corresponding side strap 20′ defining the corresponding opening 231′ in a direction toward the wearer's head, and has a distal end 222A distal from the corresponding side strap 20′. The head-engaging plate portions 221′ of each of the cushioning members 220 extend respectively and integrally from the distal ends 222A of the connecting plate portion 222′ of the corresponding cushioning members 220 toward each other, so as to define the buffering space 23′ between the connecting portions 222′ of the corresponding cushioning members 220 and between the corresponding side strap 20′ and the head-engaging plate portions 221′ of the corresponding cushioning members 220. The head-engaging plate portions 221′ of the each of side cushions 22′ define a gap 223′ therebetween. The head-engaging plate portions 221′ can also abut resiliently against the wearer's head.

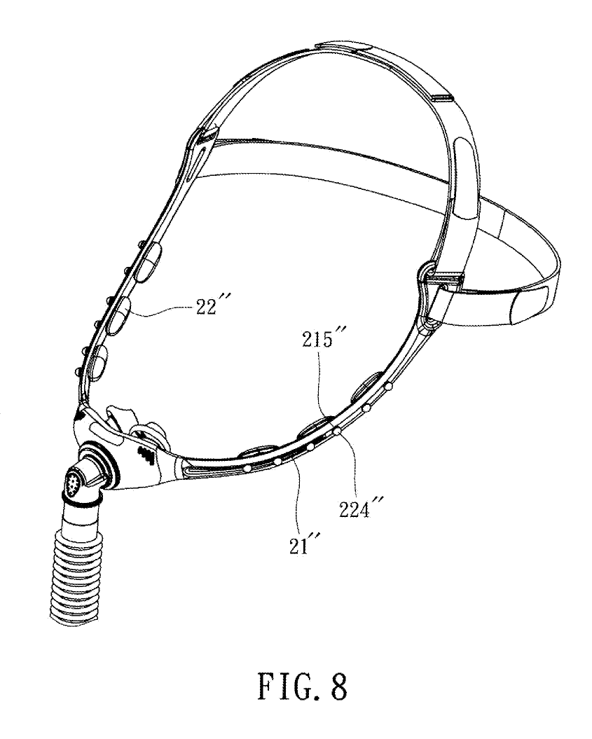

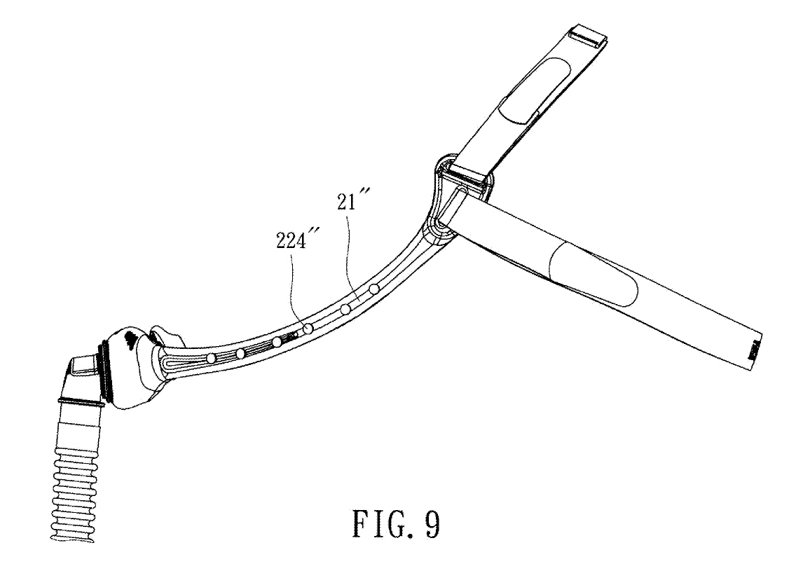

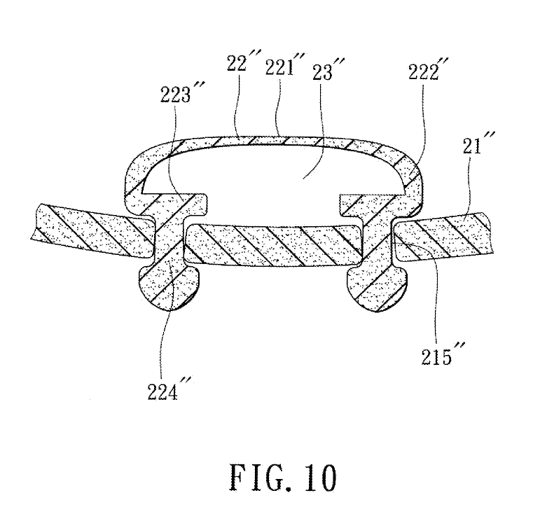

FIGS. 8, 9, and 10 show the third preferred embodiment of a breathing assistance apparatus according to this invention, which differs from the previous embodiments in that, the side cushions 22″ are connected removably to the strap bodies 21″.

In this embodiment, each of the strap bodies 21″ has a plurality of retaining holes 215″ formed therethrough. Each of the side cushions 22″ has a head-engaging portion 221″, a loop-shaped connecting portion 222″ extending, integrally from the head-engaging portion 221″ toward the corresponding strap body 21″, a loop-shaped plate portion 223″ having an outer periphery connected integrally to the connecting portion 222″, and a plurality of retaining tongues 224″ extending perpendicularly from the loop-shaped plate portion 223″. The retaining tongues 224′ of the side cushions 22″ are confined respectively within the retaining holes 215″ in the strap bodies 21. The head-engaging portion 221″, the loop-shaped connecting portion 222″, and the loop-shaped plate portion 223″ of each of the side cushions 22″ define cooperatively the buffering space 23″ thereamong.

In view of the above, due to the presence of the side cushions 22, 22′, 22″, the breathing assistance apparatus can be worn comfortably on the wearer. Thus, the object of this invention is achieved.

With this invention thus explained, it is apparent that numerous modifications and variations can be made without departing from the scope and spirit of this invention. It is therefore intended that this invention be limited only as indicated by the appended claims.

I claim:

1. A breathing assistance apparatus adapted to deliver gases to a wearer, said breathing assistance apparatus comprising:
a main body unit having a main body and two side straps connected to said main body, each of said side straps having a strap body, at least one side cushion projecting from said strap body and adapted for contact with the wearer's head, and a buffering space disposed within said side cushion;
a head strap unit connected to said side straps;
a nasal prong unit mounted to and disposed behind said main body of said main body unit; and
an intake unit mounted to and disposed in front of said main body of said main body unit.

2. The breathing assistance apparatus as claimed in claim 1, wherein said strap body of each of said side straps of said main body unit is formed with an opening, and said side cushion of each of said side straps of said main body unit has a head-engaging portion, end a loop-shaped connecting portion that is connected integrally between an outer periphery of said head-engaging portion and a wall of a corresponding one of said side straps defining said opening in said strap body of the corresponding one of said side straps, such that said buffering space is disposed inside said loop-shaped connecting portion.

3. The breathing assistance apparatus as claimed in claim 1, wherein said strap body of each of said side straps of said main body unit is formed with an opening, and said side cushion of each of said side straps of said main body unit has two spaced-apart cushioning members, each of said cushioning members having a head-engaging plate portion and a connecting plate portion, each of said connecting plate portions of said cushioning members extending integrally from a wall of a corresponding one of said side straps defining said opening in the corresponding one of said side straps in a direction toward the wearer's head and having a distal end distal from the corresponding one of said side straps, said head-engaging plate portions of each of said side cushions extending respectively and integrally from said distal ends of said connecting plate portions of a corresponding one of said side cushions toward each other, so as to define said buffering space between said connecting portions of the corresponding one of said side cushions and between the corresponding one of said side straps and said head-engaging plate portions of the corresponding one of said side cushions.

4. The breathing assistance apparatus as claimed in claim 1, wherein said strap body of each of said side straps of said main body unit has a plurality of retaining holes formed therethrough, and said main body unit includes a plurality of said side cushions, each of said side cushions having a head-engaging portion, a loop-shaped connecting portion extending integrally from said head-engaging portion toward a corresponding one of said side straps, and a plurality of retaining tongues connected integrally to said connecting portion, said retaining tongues of said side cushions being confined respectively within said retaining holes in said side straps.

5. The breathing assistance apparatus as claimed in claim 4, wherein each of said side cushions further has a loop-shaped plate portion that has an outer periphery connected integrally to said connecting portion of a corresponding one of said side cushions, such that said head-engaging portion, said loop-shaped connecting portion, and said loop-shaped plate portion of each of said side cushions define cooperatively said buffering space thereamong, each of said retaining tongues extending perpendicularly from said loop-shaped plate portion of a corresponding one of said side cushions.

6. The breathing assistance apparatus as claimed in claim 1, wherein said main body of said main body unit has a rear wall connected to said side straps, a front wall disposed in front of said rear wall, and a connecting wall connected between said front and rear walls and cooperating with said front and rear walls to define a chamber thereamong, said rear wall having an insert hole in spatial communication with said chamber, said front wall having a coupling hole in spatial communication with said chamber, said nasal prong unit including a main plate inserted into said rear wall and aligned with said insert hole, and two nasal prongs disposed on said main plate and in spatial communication with said chamber, said intake unit being mounted into said coupling hole in said front wall.

7. The breathing assistance apparatus as claimed in claim 6, wherein said rear wall of said main body unit further has a rear side surface formed with an insert groove that is disposed behind and in spatial communication with said insert hole, that has a width reducing outwardly, and that permits said main plate to be confined therein.

8. The breathing assistance apparatus as claimed in claim 1, wherein said main body unit is made of silicone gel, and said strap body of each of said side straps has a first through hole and a second through hole, said head strap units having a first head strap extending through said first through holes in said side straps, and a second head strap extending through said second through holes in said side straps.

9. The breathing assistance apparatus as claimed in claim 1, wherein said intake unit includes a tube-sleeving member inserted into said coupling hole, and an intake tube having an end permitting said tube-sleeving member to be sleeved fixedly thereon.
